(19) United States
(12) Patent Application Publication
(10) Pub. No.: US 2026/0216407 A1
(43) Pub. Date: Jul. 30, 2026

(54) PRODUCTION AND USE OF BACTERIAL CELLULOSE IN PURE FORM OR BY IMPREGNATION OF VARIOUS AGENTS AND PRODUCED IN SPHERICAL FORM FOR BONE REGENERATION, ALONE AND IN COMBINATION WITH VARIOUS GRAFT MATERIALS

(71) Applicant: ATATÜRK ÜNIVERSITESI REKTÖRLÜGÜ BILIMSEL ARASTIRMA PROJELERI ( BAP ) KOORDINASYON BIRIMI, Erzurum (TR)

(72) Inventors: SONGUL COMERT KILIC, Erzurum (TR); MURAT OZDAL, Erzurum (TR)

(73) Assignee: ATATÜRK ÜNIVERSITESI REKTÖRLÜGÜ BILIMSEL ARASTIRMA PROJELERI ( BAP ) KOORDINASYON BIRIMI, Erzurum (TR)

(21) Appl. No.: 18/293,388

(22) PCT Filed: Sep. 11, 2023
(86) PCT No.: PCT/TR2023/050939

§ 371 Date: Jan. 30, 2024

(51) Int. Cl. A61L 27/46; A61L 27/20; A61L 27/52; A61L 27/54; C12P 19/04; C12R 1/01

(52) U.S. Cl. CPC A61L 27/46; A61L 27/20; A61L 27/52; A61L 27/54; C12P 19/04; A61L 2430/02; C12R 2001/01

(57) ABSTRACT

Although this invention is primarily designed for various types of bone defects in dentistry, it can also be used in the medical field for various types of bone defects. It can be used in maxillary sinus augmentations, various intraosseous defect areas, and defect areas after intraosseous cyst surgeries, periimplantitis bone defects, and vertical/horizontal bone augmentations.

### FIELD OF THE INVENTION

Although this invention is primarily designed for various types of bone defects in dentistry, it can also be used in the medical field for various types of bone defects. It can be used in maxillary sinus augmentations, various intraosseous defect areas, bone defect areas after intraosseous cyst surgeries, periimplantitis bone defects, and vertical/horizontal bone augmentations.

### BACKGROUND OF THE INVENTION

In implant surgeries, insufficient bone areas make patient intervention difficult and require additional surgical procedures. Especially, the commercially produced grafts used in these surgeries are imported from abroad, creating significant costs for the patient and the country. Using autogenous grafts creates a second surgical field and is not tolerated and desired by the patient. The insufficient autogenous grafts in large defect areas force us to use other graft types. Besides the cost, these products cannot regenerate as fast as autogenous grafts and cannot reach a load-bearing level in the early period. For this reason, it has been developed to shorten the regeneration time of these graft materials by reducing the amount of material to be used and to provide better ossification, especially to provide earlier prosthetic loading of implants. The subantral space created in maxillary sinus augmentations and the bone defect areas that develop after cyst enucleation is usually filled with graft materials, and prosthetic loading can be performed after six months at the latest. In order to maintain the space created in the relevant area, coarse grain grafts are usually placed at a rate of 2 cc or more, and these grafts do not protect the space in which they are placed very well over time and may resorb. Here, we expect cellulose to form bone bridges between the graft particles due to the combined application of cellulose in particulate form and graft material with particles of similar size by mixing homogeneously. This will also reduce the graft cost by reducing the graft used.

We think that it can be sufficient alone by being homogeneously distributed in the subantral area and around the implant and in various other intraosseous defect areas without being mixed with the graft, even in more limited cases of augmentation of bacterial cellulose alone or impregnated with bioactive agents, for example, in maxillary sinus augmentations performed simultaneously with the implant.

Mixing and using bacterial cellulose with the graft by impregnating it with bioactive agents will increase the bone bridges between the graft particles and provide a better quality ossification in a shorter time. When cellulose is used both alone and mixed with graft material with a slow-prolonged release of bioactive agents absorbed into bacterial cellulose, we think that it will make an additional significant positive contribution to bone regeneration, and this long-term slow release will contribute to the early period of ossification as well as to the late period of ossification.

In addition to other bone graft materials, this application can be performed with autogenous grafts to provide a better ossification with the limited amount of autogenous bone used. This is also true for other types of defects.

The purpose of this application is to reduce the cost of graft by using fewer grafts in the defect areas, to protect the space of the area to be grafted in the defect area, to shorten the ossification process of the bone to be loaded, and to provide a better ossification, to ensure the continuity of this ossification quality after loading. Another reason for choosing bacterial cellulose is its high biocompatibility.

Current methods for bacterial cellulose production comprise static and stationary phase cultures. The static culture method results in the deposition of a gelatinous cellulose membrane on the surface of the medium, while the shaking culture method produces pellet or sphere-like cellulose. The choice of the method depends on the applications of the bacterial cellulose as well as the required physical, morphological, and mechanical properties.

The two main problems with static culture systems are high cost and low production rate. The use of an agitated culture has been proposed to solve these problems. Oxygen is directly related to bacterial cellulose production and is known to be a significant disadvantage of the static culture method. The basic idea behind the design of the shaking culture is to increase oxygen delivery to the bacteria during culture.

A range of bacterial cellulose-like wound dressings is commercialized under the trademarks Nanoderm™, Bionext®, Membracell®, Suprasorb® X, Biofill®, Gengiflex®, Xcell® etc. Nanoderm™ wound dressing is used for skin regeneration. Nanoderm™ Ag (Axcelon Dermacare Inc, 2020) for treating infected wounds. Axcelon Dermacare Inc. has developed many bacterial cellulose-based medical-use products (Axcelon Dermacare Inc, 2020), including contact lenses, vascular grafts, and artificial tympanic membranes. These materials are in the form of membranes, and no product is produced in spherical form.

It has been determined that bacterial cellulose produced in static culture is perfectly biocompatible with bone and connective tissue in subcutaneous implant applications (Märtson et al., 1999). Microbial cellulose has been used in bone repair as a porous matrix combined with calcium salts (Hutchens ve ark. 2012 Patent No US20040096509A1). Bovine Serum Albumin and hydroxy apatite were used for hard tissue repair and regeneration by absorbing into bacterial cellulose membranes produced in the stationary phase (Serafica and Damien 2007 patent application Ser. No. 11/761,846).

In a study, a hydrogel was formed by adding hydroxylapatite particles to the structure of nanofibrous-tetramethylpiperidin-1-oxyl (TEMPO)-oxidized bacterial cellulose to serve as a scaffold for bone tissue engineering and these gel composites with well-developed porous structure were incubated with calvarial osteoblasts. It was determined that the cellulose form prepared in this way significantly improved cell proliferation and cell differentiation, and it was emphasized that this hydrogel is a potential candidate for use in bone tissue engineering scaffolds (Park et al., 2015). Koike et al., 2019 evaluated the effect of bacterial cellulose prepared in membrane form, loaded with bone morphogenetic protein-2 (BMP-2) without delay, on bone regeneration in rabbit frontal sinus models. Bacterial cellulose and BMP-2 were used alone or in combination in sinus models, and it was reported that the Bacterial cellulose+BMP-2 group provided significantly superior results than the other groups in all index values evaluating new bone formation. It has been stated that bacterial cellulose fills the defect area and promotes optimum bone formation by continuously releasing BMP-2. It has been claimed that the combination of bacterial cellulose+BMP-2 may be beneficial for maxillary sinus augmentations by showing increased bone regeneration (Koike et al., 2019).

There are a limited number of studies evaluating the effectiveness of hydrogels on bone regeneration by mixing with autogenous or other bone graft materials in the bone defect area (Jung et al., 2015; Thoma et al., 2017; Cha et al., 2018). However, again, there are no studies in the literature in which bacterial cellulose is used mixed with graft materials.

When the studies evaluating the use of bacterial cellulose in various bone defects are examined, it is seen that cellulose is placed in the defect area in the form of gel or membrane and placed in the defect without disrupting its integrity. In the histological examinations in these studies, it was reported that bone regeneration occurred only in the outer part of the cellulose. However, no ossification occurred in the inner parts, and the cellulose remained massively unresorbed in the defect area. However, in this study, it is thought that cellulose will be produced in particulate form (in the form of pellets) and provide a completely new ossification in the defect area by new bone bridges between these particles. In addition, as a result of the combined application of cellulose in particulate form and graft material with particles of similar size, it is thought that bone bridges between particles will provide new bone formation in an earlier process and with higher quality, and it will again provide complete ossification in the defect area. In addition to the above applications, when used both alone and mixed with the graft material, with the slow and long-term release of bioactive agents absorbed into the cellulose, it is thought that it will make an additional significant positive contribution in bone regeneration and ossification areas will be provided in the inner parts of the cellulose.

Opinions on patents encountered in the basic patent search on the subject are as follows;

### Solid Dispersoid of Rifamycin-Quinazone Coupling Molecule and Application Thereof

The invention provides a solid dispersoid of a rifamycin-quinazone coupling molecule. The components of the solid dispersoid comprise the rifamycin-kinazone coupling molecule, a high molecular carrier, a functional excipient, and a solvent, as shown in formula I. The high molecular carrier comprises one or a combination of povidone K30, povidone VA64, hydroxy propyl cellulose L, a polivinyl caprolactam-polyvinyl acetate-polyethylene glycol grafted copolymer, and polymethacrylate. Functional auxiliary material comprises one or a combination of vitamin E polyethylene glycol succinate, lauryl sodium sulfate, meglumine, and Tween 80. The formula is as shown in the description. The solid dispersoid of the rifamycin-quinazone coupling molecule can be used to prepare a drug to treat bacterial infection.

In our study, pellet bacterial cellulose will be combined with related compounds.

### Compositions for the Treatment of Gastrointestinal Inflammation

Methods are provided here to prevent or alleviate symptoms and inflammation associated with inflammatory diseases and conditions of the gastrointestinal tract, such as those of the oesophagus. Here, support materials were uses, such as xanthan gum, polyethylene glycol (e.g. 200-4500) tragacanth gum, ethyl cellulose, ethylhydroxyethyl cellulose, ethylmethyl cellulose, methyl cellulose, hydroxyethyl cellulose, hydroxyethylmethyl cellulose, hydroxy propyl cellulose, poly(hydroxyethyl methacrylate), oxypolygelatin, pectin, polygelin, povidone, propylene ether/vinyl methyl anhydride copolymer (PVM/MA), poly(methoxyethyl methacrylate), poly(methoxyethoxyethyl methacrylate), hydroxypropyl cellulose, hydroxypropylmethyl-cellulose, sodium carboxymethyl-cellulose (CMC).

### Novel Antimicrobial Substrates and Uses Thereof

In this study, antimicrobial products were produced using different types of nanoparticles (Ag, Zn, Au, Pt, Cu, Si, Bi . . . ) and support materials (polyethylene, polyimide, silicone, polyvinyl chloride, polyamide, polyester, polycarbonate . . . ).

Cellulose was not used in this study.

### Liquid Composition for Oral Cavity

The present invention has an excellent salivary-promoting effect, good appearance stability after storage at room or low temperature, minor irritation during or after use, and excellent moisture feeling. y-polyglutamic acid is an effective oral salt for dry mouth symptoms. The ratio of carboxyl methyl cellulose in the mixture is between 0.05-1%. The moisture and appearance stability of sodium carboxymethyl cellulose are further improved after use. It comprises polyhydric alcohols such as glycerin, sorbitol, ethylene glycol, propylene glycol, polyethylene glycol, maltitol, and erythritol as wetting and flavoring agents in the mixture.

Our study is related to the use of bacterial cellulose in bone regeneration.

### Monomethylvaline Compounds Capable of Conjugation to Ligands

The present invention relates to a Drug compound, and more particularly to Drug-Ligand Conjugates, Drug-Linker Compounds and compositions containing them, and methods for using the same to treat cancer, which is an autoimmune disease. The present invention also relates to antibody-drug conjugates, compositions containing them, and methods of treating cancer, an autoimmune disease, or an infectious disease.

Cellulose was not used in this study.

### Molecular Vaccines for Infectious Disease

The present invention relates to methods for forming pharmaceuticals, that is, vaccine components characterized by multimerization domains and bound biologically active molecules, and their use in preparing vaccines containing pharmaceuticals, either alone or in combination with other molecules. The individual molecules of the structure can be linked to each other or the multimerization domain(s) by covalent or non-covalent bonds, directly or via linkers. The invention also relates to using such preparations in vaccine medium intended to function as preventive/prophylactic or therapeutic vaccines in humans and animals. Examples of polysaccharides useful in the present invention comprise cellulose materials, materials of vegetable or animal origin, including hemicellulose, alkyl cellulose, hydroxyalkyl cellulose, carboxymethylcellulose, sulfoethylcellulose, starch, xylan, amylopectin, chondroitin, hyaluronate, heparin, mannan, xanth.

Cellulose was not used in the study.

### Bioresorbable Calcium-Deficient Hydroxyapatite Hydrogel Composite

The present invention provides a composite material comprising oxidized bacterial cellulose and calcium-deficient hydroxy apatite, and methods for preparing the composite material. The composite material is useful as a bone graft material. In another embodiment, the invention provides a mammal tissue repair method. The method involves placing the composite material into cartilage or bone tissue. The bacterial cellulose samples used in this study were 6 cm in diameter and 3 mm thick.

In our study, bacterial celluloses in pellet form will be used.

### Methods of Making Spheroids Including Biologically-Relevant Materials

Methods of making a spheroid or sphere are provided such that a suspension containing one or more biologically relevant materials dispersed in a biocompatible medium is first produced. A drop of the suspension is then obtained by contacting the droplet with a surface of the salt solution in a controlled-manner to obtain a sphere of the desired size reproducibly and containing the desired amount of biologically relevant material. Exemplary hydrogels of the presently disclosed subject matter may consist of polymeric materials comprising: alginate, collagen (including collagen types I and VI), elastin, keratin, fibronectin, proteoglycans, glycoproteins, polylactide, polyethylene glycol, poly caprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyhydrides, polyacetates, polyhydrides, polyamino acids carbohydrates, polysaccharides and modified polysaccharides and their derivatives and copolymers, as well as inorganic materials such as bioactive glass, ceramics, silica, alumina, calcite, hydroxy apatite, calcium phosphate, bone, and combinations of all these. Cellulose was not used in this study.

Since the spheroid bacterial celluloses obtained in our study will be produced in an agitated culture, they will form naturally/spontaneously.

### Bone Graft Substitute

The present invention relates to a porous implant mass composition for treating defects in living organisms, such as bone defects and extraction wounds, and the production thereof. More specifically, it concerns a newly optimized geometry and pore structure that allows for a mechanically stable yet highly porous bone graft substitution. Examples of porogens comprise: polysaccharides such as cellulose, microcrystalline cellulose, hydroxypropylcellulose, methylcellulose, hydroxypropyl methylcellulose, hydroxypropylmethylcellulose acetate succinate (AQOAT), ethylcellulose, carboxymethylethylcellulose, cellulose propylcarcetylalcelate and their derivatives; starch, processed starch; sodium starch glycolate, pregelatinized starch. Examples can also be synthetic polymers such as polymethylmethacrylate, polyethylene, polypropylene, polystyrene, polyvinylchloride, polyvinylpyrrolidone, polyvinyl alcohol, silicones, polylactides and polyglycolides, and copolymers made from them.

The cellulose samples used in this study are 300-350 μm in size, compressed at different pressures, and the obtained samples are suggested to be used as graft material.

In our study, bacterial celluloses in spherical form will be obtained at the end of incubation.

### Minimal Tissue Attachment Implantable Materials

The invention provides a method for minimizing tissue adhesion at an injured site; the method involves applying a biocellulose material to the injured area, thereby minimizing the adhesion of tissues to the injured area, wherein the biocellulose material is at least partially dried. Another embodiment provides an implantable material that effectively inhibits cell adhesion and has desirable mechanical properties. The study produced bacterial cellulose in the static phase and treated it with oxidizing agents (hydrogen peroxide, sodium periodate, nitrogen tetroxide).

In our study, small spherical bacterial cellulose will be produced in the agitated phase.

### Poly(Vinyl Alcohol)-Bacterial Cellulose Nanocomposite

The present invention relates to composite materials formed from hydrogel and cellulose, and more particularly, the present invention relates to novel types of poly(vinyl alcohol)-bacterially produced cellulose composites suitable for soft tissue replacement and controlled release. Hydrogel was produced using PVA 5-15% and bacterial cellulose (produced in static phase) ratio of 0.15-0.5% in the mixture.

In our study, small spherical bacterial celluloses will be produced in the agitated phase, and polymer materials will not be used as additional support material.

### Bone Graft and Biocomposite for Prosthetic Dentistry

The invention relates to bone grafts and biocomposites made from a base material comprising tricalcium phosphate granules and an absorbable polymer or copolymer. In particular, the invention relates to methods and compositions for accelerating and improving the healing of bone and soft tissue lesions during prosthetic dentistry. The graft material used in the study is a copolymer of tricalcium diphosphate and biodegradable polylactic acid and poly glycolic acid.

In our study, biodegradable bacterial cellulose will be used.

### Three-Dimensional Bioprinting of Biosynthetic Cellulose (BC) Implants and Scaffolds for Tissue Engineering

The present invention relates to biomedical implants and devices, tissue engineering, regenerative medicine, and healthcare products. More particularly, embodiments of the present invention relate to systems and methods for producing and controlling the 3-D architecture and morphology of nano-cellulose biomaterials produced by bacteria using novel biofabrication processes such as 3-D Bioprinting. While the craniofacial bone grafts used in the study were 10 cm in diameter and 5 mm thick, the breast implants were 5-20 cm in diameter and 10 cm thick.

Spherical spherical form bacterial cellulose will be used as graft material in our study.

### Anisotropic Hydrogels

In this study, polymers such as Poly(vinyl alcohol) can be used as hydrogels in other medical uses such as tissue reconstruction, vascular regeneration, cartilage, stents, contact lenses, bandages, etc.

Spherical spherical form bacterial cellulose will be used as graft material in our study.

### Degradable Biomolecule Compositions

This study provides methods and materials related to degradable biomolecule compositions. For example, methods and materials are provided for compositions with one or more biomolecules and one or more biomolecule degrading enzymes capable of degrading one or more biomolecules of the composition. In some cases, the degradable biomolecule compositions provided herein are as wound dressings to facilitate wound healing, as scaffolds or tissue matrices to promote tissue growth or regeneration, as bulking agents to temporarily bulk the tissue, and to provide degradable compositions.

This study differs from our study in that it is a biodegrading enzyme.

### Composites Containing Polypeptides Attached to Polysaccharides and Molecules

This study provides methods and materials for composites containing polypeptides attached to polysaccharides or molecules. For example, it relates to producing composite materials containing polypeptides (e.g., casein polypeptides) together with polysaccharides (e.g., cellulose) or molecules (e.g., calcium-containing molecules such as calcium phosphate and calcium carbonate).

In our study, spherical bacterial celluloses will be used as graft material.

### Implantable Microbial Cellulose Materials for Hard Tissue Repair and Regeneration

The present invention relates to polysaccharide materials and, more particularly, microbial cellulose-containing materials with implantation properties suitable for repairing or replacing hard tissue. The invention also relates to implantable microbial cellulose as a bone cavity filler and as a carrier vehicle for the delivery of active substances to repair or regenerate hard tissue. The invention also relates to implantable microbial cellulose as a bone cavity filler and as a carrier vehicle for the delivery of active substances to repair or regenerate hard tissue. The study used bacterial cellulose produced in the static phase by impregnating it with different biological agents.

Our study will use small spherical bacterial celluloses in the agitated phase.

### Oxidized Microbial Cellulose and Use Thereof

This invention relates to oxidized microbial cellulose, production methods, and medical and surgical applications. The invention provides a method for making oxidized microbial cellulose by oxidation of microbial cellulose with a periodate solution. The invention also compared different oxidation methods. Oxidized microbial cellulose obtainable by these methods can be provided. Oxidized microbial cellulose could be used in therapy and surgery as a wound dressing, implantable tissue replacement, tissue engineering matrix, or anti-adhesion device.

### Methods of Bonding or Modifying Hydrogels Using Irradiation

The present invention provides methods and processes for bonding or attaching hydrogels using irradiation techniques to suitable materials such as soft tissues, elastomers, and hydrogel surfaces. The present invention also provides methods and processes for modifying hydrogel products by creating cross-linked regions in these hydrogels using these irradiation techniques.

### Blend Hydrogels and Methods of Making

The present invention provides a blend of water-swelling materials and hydrogels suitable for biomedical or other applications. The blend contains water-swellable materials and hydrogels, at least one hydrophilic polymer, and at least one other polymer or oligomer having both hydrophobic and hydrophilic repeating units, wherein the blend phase separates and is opaque and immiscible in the presence of water. This mixture also describes methods of making water-swelling materials and hydrogels.

### DEFINITION OF THE INVENTION

Said invention eliminates the disadvantages described in the state of the art and fulfills the needs.

Although this invention is primarily designed for various types of bone defects in dentistry, it can also be used in the medical field for various types of bone defects. It can be used in maxillary sinus augmentations, various intraosseous defect areas, bone defect areas after intraosseous cyst surgeries, periimplantitis bone defects, and vertical/horizontal bone augmentations.

The main advantages of said invention are as follows;
1. Prevent early resorption of the graft in the defect area and to protect the gap,
2. Reducing the cost by reducing the excess amount of graft used,
3. Creating better bone bridges between graft particles,
4. Resetting the need for graft in cases where bacterial cellulose is used alone and especially by absorbing bioactive agents,
5. Acting as a carrier for autologous products, such as PRP, for slow and long-term release by providing long-term regenerative activity of bioactive agents,
6. Mixing the graft homogeneously with the dispersed type spherical prepared form in which bioactive agents are impregnated, reduces the amount of graft used, provides early and better ossification, enabling earlier implant placement and providing earlier prosthetic loading, ensuring the continuation of quality ossification after loading, reducing the rate of failed implants and increasing the success.

All these activities are due to the application of bacterial cellulose as a carrier by impregnation with bioactive substances under agitation, creating bone bridges not only on the outer part of the cellulose but also within and between the cellulose particles by impregnation of bioactive agents, without occupying space in the defect area massively with this structure of bacterial cellulose, which is prepared in spherical form and dispersed homogeneously in the defect area. In addition, the homogeneous distribution of the cellulose form, in which the pure or bioactive agents are impregnated, between the graft particles allows the cellulose to form a bridge between the graft particles and to release the bioactive agents around each graft particle slowly and for more extended periods provides better ossification.

About the conditions that are effective for achieving the above advantages;
1. Providing bone regeneration between cellulose particles and within cellulose by homogeneously intermittent distribution of bacterial cellulose in the defect area and slow and long-term release of bioactive agents in pure or impregnated form, without mass continuity in the defect areas,
2. Providing bone regeneration between and within the graft and cellulose particles by slow and long-term release of pure or impregnated bioactive agents by mixing homogeneously with the graft particles and positioning around the graft particles,

These properties are achieved by using a different method of cellulose production and its releasing properties. Compared with bacterial cellulose prepared by static culture, sphere/pellet-like bacterial cellulose produced in agitated culture has a loose, layered, porous character and highly hydrophilic network structures that provide additional advantages. More importantly, the surface area of bacterial cellulose increases in spherical form.

The main features of bacterial cellulose are its high biocompatibility, low toxicity, environmental friendliness, and resistance to high tensile and pulling forces. Furthermore, its important advantages are its high purity, microfiber structure, high water holding capacity, high hydrophilic properties, easy sterilization, and ability to be produced in the desired shape and size.

### DESCRIPTION OF THE INVENTION

The present invention related to the production of bacterial cellulose in pure form or by impregnation of various agents and produced in spherical form for bone regeneration, alone and in combination with various graft materials, characterized in that it comprises the following process steps: growing Komagataeibacter europaeus strain MOZ (GenBank: MW131623.1) in medium of 20 g/L mannitol, 4 g/L yeast extract, 8 g/L peptone, 2.3 g/L Na2HPO4 and pH 6.0 for bacterial cellulose production and adding (20 g/L mannitol, 4 g/L yeast extract, 8 g/L peptone, 2.3 g/L Na2HPO4, 1.0 g/L citric acid and pH 6.0) to the prepared liquid culture, using the same for inoculation at a rate of 6% and incubating for six days at 30° C. in a culture shaker incubator.

### In This Invention; in Purification of Bacterial Cellulose

At the end of the incubation, the fermentation liquid will be filtered away and washed with pure water to purify the bacterial cellulose. The obtained celluloses will be placed in 0.1 M NaOH solution and kept in a water bath at 90° C. for 30 minutes to remove bacterial cells and media residues. At the end of the period, it will be washed with distilled water until the pH reaches 7 (Żywicka et al. 2018).

### In This Invention; Preparation of Bacterial Cellulose Pellets Treated With Acetic Acid

Insufficient porosity or small pore size limits the post-tissue engineering applications of bacterial cellulose. For this purpose, acetic acid is applied to increase the pore content of bacterial cellulose. Purified bacterial cellulose pellets will be placed in 1% acetic acid for 12 hours at room temperature. At the end of the period, the bacterial cellulose pellets are washed with distilled water and used in studies (Hu et al. 2014).

### In This Invention; Hydroxyapatite/Bacterial Cellulose Composite Synthesis

Hydroxyapatite (HA) is formed in bacterial cellulose hydrogel by performing various incubation cycles with calcium and phosphate solutions. Briefly, bacterial cellulose hydrogels are incubated in a CaCl₂ (11 g/L) solution at pH 4.83 under shaking in an orbital shaker for 12 h at 23° C. Bacterial cellulose hydrogels are rinsed with sterile deionized water and then incubated for 12 hours in a Na₂HPO₄ solution (8.52 g/L). The samples are then rinsed in deionized water and dried to constant weight at 60° C.

### In This Invention; Bioactive Agents/Bacterial Cellulose Composite Synthesis

Bioactive agents are impregnated to form bacterial cellulose hydrogel. The agents to be added to the bacterial cellulose are added to the sterile cellulose at the desired concentrations in a sterile manner. Bacterial cellulose hydrogels are made under agitation in an orbital shaker for 24 hours at 23° C.

The present invention uses bacterial cellulose in pure form or by impregnating various agents produced in spherical form for bone regeneration alone and combination with various graft materials. These are as follows;
1. Use in various bone defects by spherical preparation of bacterial cellulose in dispersed form,
2. Use of bacterial cellulose in various bone defects by preparing spherically dispersed cellulose and adding various bioactive agents therein,
3. Use of bacterial cellulose in various bone defects by preparing the same spherically in dispersed form and mixing it with various graft materials in particulate form,
4. Bacterial cellulose is prepared spherically in dispersed form, and various bioactive agents are added therein. This impregnated form is mixed with various graft materials in particulate form and used in various bone defects.

1. Production of bacterial cellulose in pure form or by impregnation of various agents and produced in spherical form for bone regeneration, alone and in combination with various graft materials, characterized in that, it comprises the following process steps; in bacterial cellulose production, inoculating Komagataeibacter europaeus strain MOZ (GenBank: MW131623.1) bacteria into 250 mL flasks containing 50 ml broth (20 g/L mannitol, 4 g/L yeast extract, 8 g/L peptone, 2.3 g/L Na2HPO4, 1.0 g/L citric acid and pH 6.0), and for inoculum, growing bacteria grown in a liquid medium that does not contain citric acid, and using the prepared liquid culture for inoculation into 6% fermentation medium, and incubated for six days at 30° C. in a culture shaker incubator.

2. Production of bacterial cellulose in pure form or by impregnation of various agents and produced in spherical form for bone regeneration, alone and in combination with various graft materials, characterized in that, it comprises the following process steps of: purifying the bacterial cellulose at the end of the incubation, filtering the fermentation liquid and washing the same with distilled water, placing obtained celluloses in 0.1 M NaOH solution and keeping in a water bath at 90° C. for 30 minutes to remove bacterial cells and media residues, washing with distilled water until the pH reaches seven at the end of the period.

3. Production of bacterial cellulose in pure form or by impregnation of various agents and produced in spherical form for bone regeneration, alone and in combination with various graft materials according to claim 1, characterized in that, it consists of the following process steps: placing the bacterial cellulose pellets in 1% acetic acid for 12 hours at room temperature in the preparation of bacterial cellulose pellets treated with acetic acid and washing the bacterial cellulose pellets with distilled water at the end of the period.

4. Production of bacterial cellulose in pure form or by impregnation of various agents and produced in spherical form for bone regeneration, alone and in combination with various graft materials according to claim 1, characterized in that, it comprises the following process steps; for hydroxyapatite/bacterial cellulose composite synthesis, incubating bacterial cellulose hydrogels in a solution of CaCl2 (11 g/L) at pH 4.83 under shaking in an orbital shaker for 12 hours at 23° C., rinsing bacterial cellulose hydrogels with sterile deionized water and then incubating the same for 12 hours in a Na2HPO4 solution (8.52 g/L) and drying at 60° C. to constant weight, forming in bacterial cellulose hydrogel by performing various incubation cycles with calcium and phosphate solutions.

5. Production and use of bacterial cellulose in pure form or by impregnation of various agents and produced in spherical form for bone regeneration, alone and in combination with various graft materials according to claim 1, characterized in that, the agents to be added to bacterial cellulose are added to sterile cellulose at desired concentrations. Bacterial cellulose hydrogels are made under shaking in an orbital shaker at 23° C. for 24 hours and bioactive agents are impregnated to form bacterial cellulose hydrogel.

6. Use of bacterial cellulose obtained in pure form or by impregnation with various agents and produced in spherical form, characterized in that; it is the spherical preparation of bacterial cellulose in dispersed form and its use in various bone defects.

7. The use of bacterial cellulose obtained in pure form or by impregnation with various agents and produced in spherical form is characterized in that; it uses bacterial cellulose in various bone defects by preparing spherically dispersed cellulose and adding various bioactive agents therein.

8. Use of bacterial cellulose obtained in pure form or by impregnation with various agents and produced in spherical form, characterized in that; It is the use of bacterial cellulose in various bone defects by preparing the same spherically in dispersed form and mixing the same with various graft materials in particulate form.

9. Use of bacterial cellulose obtained in pure form or by impregnation with various agents and produced in spherical form, characterized in that; bacterial cellulose is prepared spherically in dispersed form and various bioactive agents are added therein, and this impregnated form is used in various bone defects by mixing the same with various graft materials in particulate form.
